(19) United States
(12) Patent Application Publication
(10) Pub. No.: US 2019/0090939 A1
(43) Pub. Date: Mar. 28, 2019

(54) TREATMENT TOOL

(71) Applicant: Olympus Corporation, Tokyo (JP)

(72) Inventors: Kazuhiro Tanaka, Tokyo (JP); Tatsuro Yamamoto, Tokyo (JP)

(73) Assignee: Olympus Corporation, Tokyo (JP)

(21) Appl. No.: 16/198,176

(22) Filed: Nov. 21, 2018

Related U.S. Application Data
(63) Continuation of application No. PCT/JP2016/066262, filed on Jun. 1, 2016.

(51) Int. Cl. A61B 18/14; A61B 18/08

(52) U.S. Cl. CPC A61B 18/1445; A61B 2018/00994; A61B 18/085

(57) ABSTRACT

A treatment tool includes a rotary member that is rotatable in unison with an elongated member such as a sheath or shaft and an end effector around a predetermined axis of rotation with respect to a housing. The rotary member includes a first ridge and a second ridge each of which projects to an outer circumferential side. The second ridge is contiguous to a distal-end side of the first ridge. A second distance from the axis of rotation to a protrusive end of the second ridge is longer than a first distance from the axis of rotation to a protrusive end of the first ridge.

### CROSS-REFERENCE TO RELATED APPLICATION

This application is a continuation application of PCT Application No. PCT/JP2016/066262 filed on Jun. 1, 2016, which is hereby incorporated by reference in its entirety.

### TECHNICAL FIELD

The disclosed technology generally relates to a treatment tool for treating a treatment target with an end effector.

### DESCRIPTION OF THE RELATED ART

U.S. Pat. No. 5,383,888 discloses a treatment tool having an end effector for treating a treatment target. The end effector is disposed on the distal end of a shaft. The shaft is coupled to a housing that can be held. When a handle is closed or opened with respect to a grip of the handle, the space between a pair of gripping members of the end effector is closed or opened. When the space between the gripping members is closed, a treatment target such as a living tissue or the like is gripped between the gripping members. A rotary member or rotary knob is mounted on the housing such that the rotary member is rotatable about the central axis of the shaft. When a manipulating force for rotating the rotary member is applied, the shaft and the end effector are rotated about the central axis of the shaft as a predetermined axis of rotation in unison with the rotary member with respect to the housing. The angular position of the end effector about the predetermined axis of rotation is thus changed. Furthermore, the end effector is bent with respect to the shaft, i.e., the central axis of the shaft, based on a manipulation of a bending manipulator or wing member on the housing.

### BRIEF SUMMARY OF EMBODIMENTS

The present disclosure has been made in order to solve the problems described hereinbefore. It is an object of the present disclosure to provide a treatment tool which effectively prevents an end effector and a sheath or shaft from being rotated by a force acting on the end effector.

One aspect of the disclosed technology is directed to a treatment tool comprises a housing and an elongated member such as a sheath or shaft having respective proximal and distal ends. The elongated member such as a sheath or shaft is configured to be attached to the housing via the proximal end. The elongated member such as a sheath or shaft rotates around an axis of rotation with respect to the housing. An end effector is configured to be attached to the distal-end of the elongated member. A rotary member is configured to be attached to the elongated member. The rotary member rotates in unison with the elongated member and the end effector around the axis of rotation. The rotary member having at least respective first and second ridges. The at least first ridge projects to an outer circumferential side and having a first distance from the axis of rotation to a protrusive end thereof. The at least second ridge is contiguous to a distal-end side of the at least first ridge projecting to the outer circumferential side and having a second distance from the axis of rotation to a protrusive end thereof. The second distance is longer than the first distance.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a schematic view of a treatment tool according to a first embodiment, as viewed from one side of a housing thereof in its widthwise directions.

FIG. 2 is a schematic view of the treatment tool according to the first embodiment, as viewed from a side opposite the side where a grip is positioned, across a predetermined axis of rotation.

FIG. 3 is a schematic view of a rotary member according to the first embodiment, as viewed from a proximal-end side thereof.

FIG. 4 is a cross-sectional view taken alone line IV-IV of FIG. 3.

FIG. 5 is a cross-sectional view taken alone line V-V of FIG. 3.

FIG. 6 is a schematic view of a first ridge and a second ridge contiguous to the distal-end side of the first ridge according to the first embodiment, as viewed from an outer circumferential side of the rotary member.

FIG. 7 is a schematic view of the housing according to the first embodiment that is held by a hand, as viewed from one side of the housing in its widthwise directions.

FIG. 8 is a schematic view of the housing according to the first embodiment that is held by the hand, as viewed from the side opposite the side where the grip is positioned, across the predetermined axis of rotation.

FIG. 9 is a schematic view illustrating the manner in which an index finger abuts against a second ridge according to the first embodiment, as viewed from the outer circumferential side of the rotary member.

FIG. 10 is a schematic view illustrating the structure of a distal-end portion of a treatment tool according to a first modification.

FIG. 11 is a schematic view illustrating the structure of a distal-end portion of a treatment tool according to a second modification.

FIG. 12 is a schematic view illustrating the structure of a distal-end portion of a treatment tool according to a third modification.

FIG. 13 is a perspective view of a housing 2 and nearby structures of a treatment tool according to a reference example.

FIG. 14 is a perspective view of the housing and nearby structures of the treatment tool according to the reference example, as viewed from a direction different from FIG. 13.

FIG. 15 is a view of the housing and nearby structures of the treatment tool according to the reference example, as viewed from a distal-end side thereof.

FIG. 16 is a view of the housing and nearby structures of the treatment tool according to the reference example, as viewed from a proximal-end side thereof

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

### First Embodiment

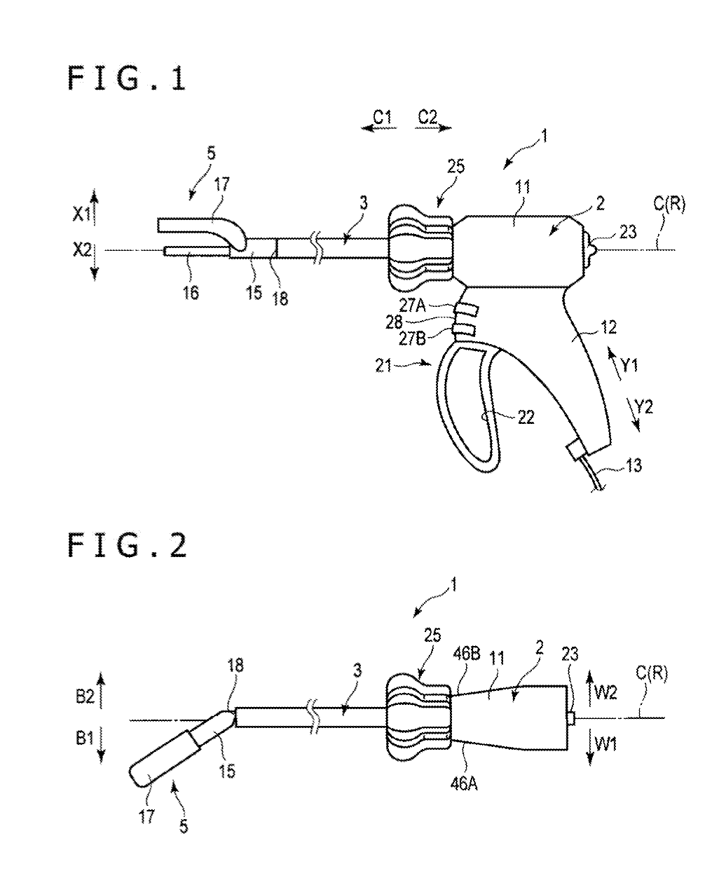

A first embodiment of the present disclosure will be described with reference to FIGS. 1 through 9. FIGS. 1 and 2 are views illustrating a treatment tool or gripping treatment tool 1 according to the present embodiment. As illustrated in FIGS. 1 and 2, the treatment tool 1 has a longitudinal axis C. Of the treatment tool 1, one side in a direction along the longitudinal axis C will be referred to as a distal-end side or arrow C1 side, and a side opposite the distal-end side will be referred to as a proximal-end side or arrow C2 side.

The treatment tool 1 includes a housing 2 that can be held, an elongated member defined by a shaft or sheath 3 coupled to a distal-end side of the housing 2, and an end effector 5 disposed on a distal-end portion of the sheath or shaft 3. The elongated member such as the sheath or the shaft 3 extends along the longitudinal axis from a proximal-end side to a distal-end side. The shaft 3 has a central axis that is substantially coaxial with the longitudinal axis C. The shaft 3 has a side extending toward the housing 2 as the proximal-end side and a side extending toward the end effector 5 as the distal-end side. The elongated member such as the sheath or shaft 3 is rotatable about its central axis with respect to the housing 2. In other words, the central axis of the sheath or shaft 3 is used as an axis of rotation R about which the shaft 3 is rotatable about the housing 2.

The housing 2 includes a housing body 11 extending along the longitudinal axis C, i.e., the axis of rotation R of the shaft 3, and a grip or fixed handle 12 extending from the housing body 11 in directions transverse to the axis of rotation R, i.e., the directions indicated by the arrows Y1, Y2. The grip 12 is disposed in a region spaced from the axis of rotation R, i.e., the longitudinal axis C. A cable 13 has an end connected to the grip 12. The other end of the cable 13 is connected to an energy controller, not depicted. The directions that are transverse to the longitudinal axis C, i.e., the axis of rotation R, or substantially perpendicular thereto and that are also transverse to the direction in which the grip 12 extends, or substantially perpendicular thereto, will also be referred to as widthwise directions of the housing 2, i.e., the directions indicated by the arrows W1, W2. FIG. 1 is a view of the treatment tool 1 as viewed from one side in a widthwise direction of the housing 2, i.e., an arrow W1 side. FIG. 2 is a view of the treatment tool 1 as viewed from a side opposite the side where the grip 12 is positioned, across the axis of rotation R, i.e., the longitudinal axis C.

The end effector 5 is rotatable in unison with the shaft 3 about the axis of rotation R with respect to the housing 2. The end effector 5 is also bendable with respect to the elongated member such as the shaft 3, i.e., the axis of rotation R. When the end effector 5 rotates, the angular position of the end effector 5 about the axis of rotation R is changed. The directions in which the end effector 5 is bendable, i.e., the directions indicated by the arrows B1, B2, are transverse to the axis of rotation R or substantially perpendicular thereto. The end effector 5 includes a relay member 15, a first gripping member 16, and a second gripping member 17. The relay member 15 is attached to the distal end of the elongated member such as the shaft 3 such that the relay member 15 is bendable with respect to the shaft 3. In other words, a bendable joint 18 is formed between the shaft 3 and the relay member 15. On the end effector 5, the space between the gripping members 16, 17 can selectively be opened and closed. The directions in which the gripping members 16, 17 are opened and closed, i.e., the directions indicated by the arrows X1, X2, are transverse to the axis of rotation R and are also transverse to the directions in which the end effector 5 is bendable.

According to an embodiment, one of the gripping members 16, 17 is integral with or fixed to the relay member 15. The other of the gripping members 16, 17 is angularly movably attached to the relay member 15. According to another embodiment, both the gripping members 16, 17 are angularly movably attached to the relay member 15. According to still another embodiment, a rod, not depicted, extends from within the relay member 15 toward the distal-end side, and a portion of the rod that projects from the relay member 15 toward the distal-end side is used as one of the gripping members 16, 17. The other of the gripping members 16, 17 is angularly movably attached to the relay member 15.

A handle or movable handle 21 is angularly movably mounted on the housing 2. When the handle 21 is angularly moved with respect to the housing 2, the handle 21 is opened or closed with respect to the grip 12. In other words, the handle 21 is openable and closable with respect to the grip 12. The handle 21 has a force applying portion 22 to which a manipulating force for opening or closing the handle 21 with respect to the grip 12 is applicable. According to the present embodiment, since the treatment tool 1 is pistol-shaped, the force applying portion 22 is positioned on one side of the axis of rotation R, i.e., the longitudinal axis C, where the grip 12 is positioned, and on the distal-end side with respect to the grip 12. When the handle 21 is opened and closed with respect to the grip 12, the handle 21 is moved in directions substantially parallel to the longitudinal axis C. When a manipulating force is applied to the force applying portion 22, opening or closing the handle 21 with respect to the grip 12, a movable member, not depicted, extending in the shaft 3 moves along the longitudinal axis C, i.e., the axis of rotation R, with respect to the shaft 3 and the housing 2. At least one of the gripping members 16, 17 is now angularly moved with respect to the relay member 15, opening or closing the space between the gripping members 16, 17.

A bending dial 23 as a bending manipulation input portion is mounted on the housing 2. When a manipulation input is applied through the bending dial or bending manipulation input portion 23 by angularly moving the bending dial 23, for example, a bending wire, not depicted, extending in the shaft 3 moves along the longitudinal axis C, i.e., the axis of rotation R, with respect to the shaft 3 and the housing 2. The end effector 5 is now bent with respect to the elongated member such as the shaft 3. The bending dial 23 may be rotatable in unison with the shaft 3 and the end effector 5 about the axis or rotation R with respect to the housing 2, or may not be rotatable in unison with the shaft 3 and the end effector 5 about the axis or rotation R. According to the present embodiment, the bending dial 23 is mounted on a proximal-end face of the housing body 11. However, the position of the bending dial 23 is not limited to the illustrated position. A bending manipulation input portion such as a bending dial 23 or the like may be mounted on an outer surface of the housing body 11 which faces a side opposite to the side of the axis of rotation R, i.e., the longitudinal axis C, where the grip 12 is positioned.

A rotary member or rotary knob 25 is mounted on the distal-end side of the housing body 11. The shaft 3 is mounted on the housing 2 while being inserted from the distal-end side into the rotary member 25 and the housing body 11. The rotary member 25 is fixed to the shaft 3. The rotary member 25 is rotatable in unison with the shaft 3 and the end effector 5 about the axis of rotation R with respect to the housing 2. According to the present embodiment, a manipulating force tending to rotate the shaft 3 and the end effector 5 about the axis of rotation R is applied to the rotary member 25 that is used as a rotational manipulation input member.

Manipulating buttons 27A, 27B are mounted on the housing 2. When each of the manipulating buttons 27A, 27B is pressed, a manipulation input is entered therefrom. The housing 2 has, on an outer surface thereof, a mount surface 28 that faces the distal-end side at a position between the force applying portion 22 of the handle 21 and the rotary member 25. The manipulating buttons 27A, 27B are disposed on the mount surface 28. Consequently, each of the manipulating buttons 27A, 27B is spaced from the axis of rotation R, i.e., the longitudinal axis C, by a smaller distance than the force applying portion 22 of the handle 21. In other words, each of the manipulating buttons 27A, 27B is closer to the axis of rotation R. When a manipulation input is entered from the manipulating button 27A, the treatment tool 1 operates in a first mode of operation. When a manipulation input is entered from the manipulating button 27B, the treatment tool 1 operates in a second mode of operation that is different from the first mode of operation. One of the manipulating buttons 27A, 27B may not be disposed on the mount surface 28, but may be disposed on an area of the outer surface of the housing 2 which faces one side in the widthwise directions. In this case, too, each of the manipulating buttons 27A, 27B is spaced from the axis of rotation R by a smaller distance than the force applying portion 22 of the handle 21.

In the first mode of operation and the second mode of operation, for example, treatment energy is applied to a treatment target gripped between the gripping members 16, 17 in respective states that are different from each other. According to an embodiment, in the first mode of operation, high-frequency electric energy is supplied from an energy controller to respective electrodes, not depicted, of the gripping members 16, 17. A high-frequency current then flows through the gripped treatment target between the gripping members 16, 17, causing the gripped treatment target to coagulate using the high-frequency current as treatment energy. In the second mode of operation, a heat generator included in the end effector 5, i.e., at least one of the gripping members 16, 17, is supplied with electric energy such as direct-current electric power or alternating-current electric power different from the high-frequency electric energy described hereinbefore from the energy controller. The heat generator then generates heat, causing the gripped treatment target to cut open and coagulate using the generated heat as treatment energy. At this time, a high-frequency current may be supplied to flow through the gripped treatment target.

According to another embodiment, instead of the heat generator, an ultrasonic transducer, not depicted, is disposed in the relay member 15 or the like. In the second mode of operation, for example, the ultrasonic transducer is supplied with electric energy such as alternating-current electric power having a predetermined frequency that is different from the high-frequency electric energy described hereinbefore from the energy controller. The ultrasonic transducer then generates ultrasonic vibrations, which are applied as treatment energy to the treatment target. According to still another embodiment, the treatment tool 1 includes an electric motor, not depicted. In the second mode of operation, for example, drive electric power is supplied to the electric motor. The electric motor is energized to stick a staple into the treatment target gripped between the gripping members 16, 17.

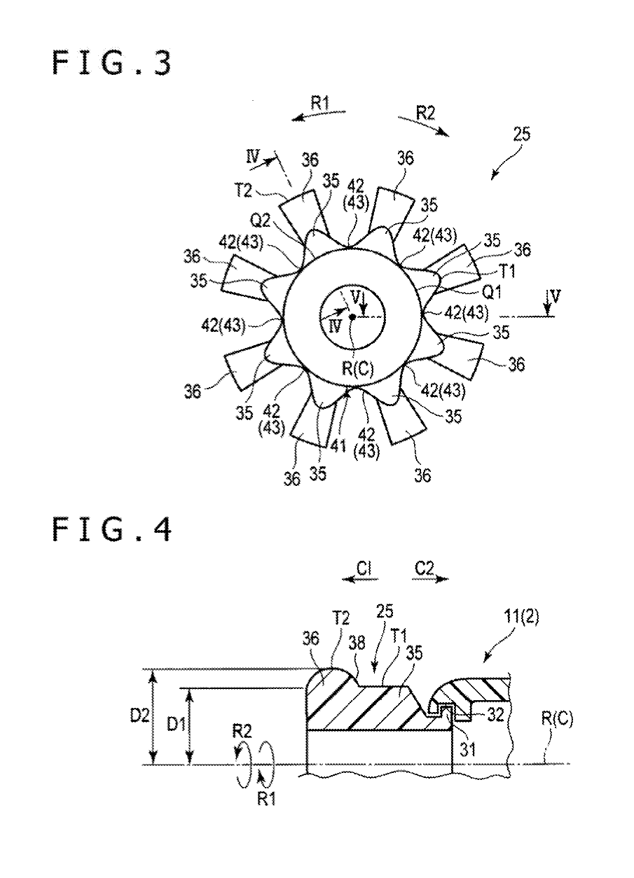

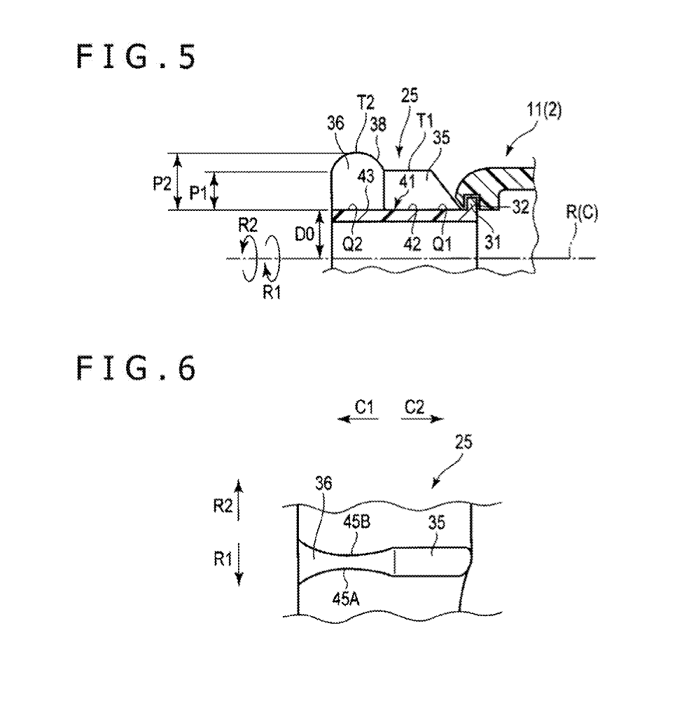

FIGS. 3 through 5 are views illustrating the structure of the rotary member 25. FIG. 3 is a view of the rotary member or rotary knob 25, as viewed from the proximal-end side thereof or the arrow C2 side. FIG. 4 is a cross-sectional view taken alone line IV-IV of FIG. 3, and FIG. 5 is a cross-sectional view taken alone line V-V of FIG. 3. As depicted in FIGS. 4 and 5, the rotary member 25 has an engaging ledge 31 projecting from the proximal-end of an outer circumferential surface thereof toward an outer circumferential side thereof. The engaging ledge 31 extends fully circumferentially around the axis of rotation R. The housing body 11 has an engaging recess 32 defined in a distal-end portion of an inner circumferential surface thereof and being recessed toward an outer circumferential side thereof. The engaging recess 32 extends fully circumferentially around the axis of rotation R. The rotary member 25 has a proximal-end portion inserted in the housing body 11, with the engaging ledge 31 engaging in the engaging recess 32. The engaging ledge 31 is movable in the engaging recess 32 around the axis of rotation R. Since the engaging ledge 31 engages in the engaging recess 32, the rotary member 25 is mounted on the housing 2 so as to be rotatable about the axis of rotation R.

As illustrated in FIGS. 3 through 5, the rotary member 25 has a plurality of, i.e., eight in the present embodiment, first ridges 35 and a plurality of, i.e., eight in the present embodiment, second ridges 36 disposed on the outer circumferential surface thereof and projecting toward the outer circumferential side. The first ridges 35 and the second ridges 36 are positioned on the distal-end side of the engaging ledge 31 and are exposed outwardly. The first ridges 35 are spaced apart from each other around the axis of rotation R, i.e., the longitudinal axis C. The second ridges 36 are spaced apart from each other around the axis of rotation R. The number of the first ridges 35 is the same as the number of the second ridges 36. Each of the second ridges 36 is contiguous to the distal-end side of a corresponding one of the first ridges 35. According to the present embodiment, each of the second ridges 36 forms part of the distal end of the rotary member 25. The first ridges 35 should preferably be disposed symmetrically around the axis of rotation R, i.e., the longitudinal axis C. The second ridges 36 should preferably be disposed symmetrically around the axis of rotation R. In this case, the first ridges 35 are disposed at substantially equal spaced intervals around the axis of rotation R. The second ridges 36 are also disposed at substantially equal spaced intervals around the axis of rotation R. FIG. 4 illustrates a cross section substantially parallel to the longitudinal axis C, which extends through a first ridge, i.e., one of the first ridges 35, and a second ridge 36, i.e., one of the second ridges 36, that is contiguous to the distal-end side of that first ridge, i.e., one of the first ridges 35.

Each of the first ridges 35 has a protrusive end T1 or a first protrusive end T1. Each of the first ridges 35 has a first distance D1 in a radial direction of the rotary member 25 from the axis of rotation R, i.e., the longitudinal axis C, to the protrusive end T1. On each of the first ridges 35, the distance from the axis of rotation R is the largest at the protrusive end T1. Each of the second ridges 36 has a protrusive end T2 or a second protrusive end T2. Each of the second ridges 36 has a second distance D2 in a radial direction of the rotary member 25 from the axis of rotation R, i.e., the longitudinal axis C, to the protrusive end T2. The second distance D2 is larger than the first distance D1. Consequently, a radial step 38 or a corresponding one of steps 38 is formed between each of the first ridges 35 and a second ridge 36 or a corresponding one of the second ridges 36 that is contiguous to the distal-end side of the first ridge 35. Because of the step 38, the protrusive end T2 or the second protrusive end T2 of each of the second ridges 36 is positioned more toward the outer circumferential side, i.e., more spaced from the axis of rotation R, than the protrusive end T1 or the first protrusive end T1 of each of the first ridges 35. On each of the second ridges 36, the distance from the axis of rotation R is the largest at the protrusive end T2.

The rotary member 25 has on its outer circumferential surface a defining face 41 that defines a base Q1 or a first base Q1 of each of the first ridges 35 and a base Q2 or a second base Q2 of each of the second ridges 36. On each of the first ridges 35, the distance from the axis of rotation R is the smallest at the base Q1. On each of the second ridges 36, the distance from the axis of rotation R is the smallest at the base Q2. The defining face 41 is exposed outwardly. According to the present embodiment, the defining face 41 is of an arcuate shape about the axis of rotation R at its center, as viewed in the directions along the axis of rotation R, i.e., toward the distal-end side and the proximal-end side, or in a cross section perpendicular to the axis of rotation R. The defining face 41 extends at least in a range from the proximal end of the first ridge 35 to the distal end of the second ridge 36 in the directions along the axis of rotation R, i.e., the longitudinal axis C. The defining face 41 extends smoothly without radial steps from the proximal end to the distal end. According to an embodiment, the defining face 41 has a radial distance D0 from the axis of rotation R that is substantially uniform from the proximal end to the distal end of the defining face 41. In this case, a protruded distance P2 or a second protruded distance P2 from the base Q2 to the protrusive end T2 of each of the second ridges 36 is larger than a protruded distance P1 or a first protruded distance P1 from the base Q1 to the protrusive end T1 of each of the first ridges 35. According to another embodiment, the defining face 41 includes a distance varying portion in which the radial distance D0 from the axis of rotation R is gradually larger from the proximal end to the distal end.

In any of the embodiments, the distance D0 radially from the axis of rotation R to the defining face 41 is smaller than the first distance D1 in the range in which the first ridges 35 extend in the directions along the axis of rotation R. Therefore, each of the first ridges 35 and another first ridge 35, i.e., a corresponding one of the first ridges 35, adjacent thereto on one side, i.e., the arrow R1 side, around the axis of rotation R define therebetween a first valley 42, a corresponding one of valleys 42, that is recessed toward the inner circumferential side. In other words, in the cross section substantially perpendicular to the axis of rotation R, which extends through the first ridges 35, the first ridges 35 and the first valleys 42 are alternately disposed around the axis of rotation R, i.e., the longitudinal axis C.

The distance D0 radially from the axis of rotation R to the defining face 41 is smaller than the second distance D2 in the range in which the second ridges 36 extend in the directions along the axis of rotation R. Therefore, each of the second ridges 36 and another second ridge 36, i.e., a corresponding one of the second ridges 36, adjacent thereto on one side, i.e., the arrow R1 side, around the axis of rotation R define therebetween a second valley 43 or a corresponding one of valleys 43, that is recessed toward the inner circumferential side. In other words, in the cross section substantially perpendicular to the axis of rotation R, which extends through the second ridges 36, the second ridges 36 and the second valleys 43 are alternately disposed around the axis of rotation R, i.e., the longitudinal axis C. As described hereinbefore, each of the second ridges 36 is contiguous to the distal-end side of a corresponding one of the first ridges 35. Therefore, each of the second valleys 43 is contiguous to the distal-end side of a corresponding one of the first valleys 42. FIG. 5 illustrates a cross section substantially parallel to the longitudinal axis C, which extends through a first valley, i.e., one of the first valleys 42, and a second valley, i.e., one of the second valleys 43, that is contiguous to the distal-end side of that first valley, i.e., one of the first valleys 42. The defining face 41 may not necessarily be of an arcuate curved surface about the axis of rotation R at its center, as viewed in the directions along the axis of rotation R. According to an embodiment, the defining face 41 may be of a flat shape. Even in such a case, the defining face 41 defines the base Q1 or the first base Q1 of the first ridge 35 and the base Q2 or the second base Q2 of the second ridge 36.

FIG. 6 is a view of a first ridge 35 or one of the first ridges 35 and a second ridge 36 or one of the second ridges 36 that is contiguous to the distal-end side of the first ridge 35, as viewed from an outer circumferential side of the rotary member 25. As illustrated in FIG. 6, each of the second ridges 36 has recesses 45A, 45B defined therein that are recessed around the axis of rotation R, i.e., in the circumferential directions of the rotary member 25. On each of the second ridges 36, the recess 45A is defined in an end face thereof on one side, i.e., the arrow R1 side, around the axis of rotation R, and is recessed toward the other side, i.e., the arrow R2 side, around the axis of rotation R. On each of the second ridges 36, the recess 45B is defined in an end face thereof on the other side, i.e., the arrow R2 side, around the axis of rotation R, and is recessed toward the one side, i.e., the arrow R1 side, around the axis of rotation R. On each of the second ridges 36, each of the recesses 45A, 45B is shaped to fit over the pad of an index finger.

As illustrated in FIG. 2, the housing 2 has on its outer surface a slanted surface 46A disposed in an area facing one side, i.e., the arrow W1 side, in a widthwise direction thereof and inclined to the longitudinal axis C. The housing 2 also has on its outer surface a slanted surface 46B disposed in an area facing the other side, i.e., the arrow W2 side, in a widthwise direction thereof. Each of the slanted surfaces 46A, 46B is disposed on the distal-end portion of the housing body 11, and extends toward the distal-end side up to the distal end of the housing body 11, i.e., the boundary position between the housing 2 and the rotary member 25. Each of the slanted surfaces 46A, 46B is inclined to the axis of rotation R, i.e., the longitudinal axis C, toward inner sides in the widthwise directions gradually in a direction toward the distal-end side. In other words, the distance from the axis of rotation R or the longitudinal axis C is gradually reduced toward the distal-end side.

Next, operation and advantages of the treatment tool 1 according to the present embodiment will be described. For treating a treatment target such as a living tissue or the like using the treatment tool 1, the operator holds the housing 2 with one hand thereof, i.e., the left hand or the right hand thereof, and inserts the end effector 5 into a body cavity such as an abdominal cavity or the like. The operator then rotates the rotary member 25 to rotate the shaft 3 and the end effector 5 about the axis of rotation R, and operates the bending dial 23 to bend the end effector 5 with respect to the shaft 3, thereby adjusting the position and posture of the end effector 5 in the body cavity. After having adjusted the position and posture of the end effector 5 in a manner to place the treatment target between the gripping members 16, 17, the operator closes the handle 21 with respect to the grip 12, closing the space between the gripping members 16, 17. The treatment target is now gripped between the gripping members 16, 17. With the treatment target being gripped, the operator presses the manipulating button 27A or 27B to enter a manipulation input, operating the treatment tool 1 in a first mode of operation or a second mode of operation. Treatment energy such as a high-frequency current is applied to or a staple is stuck into the treatment target gripped as described hereinbefore.

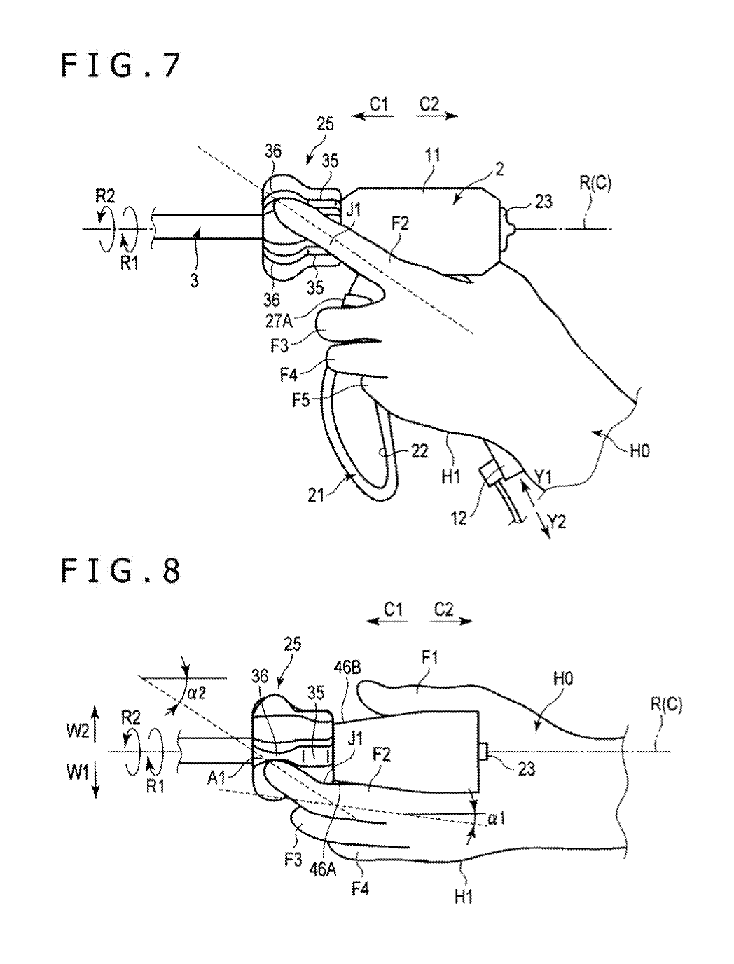

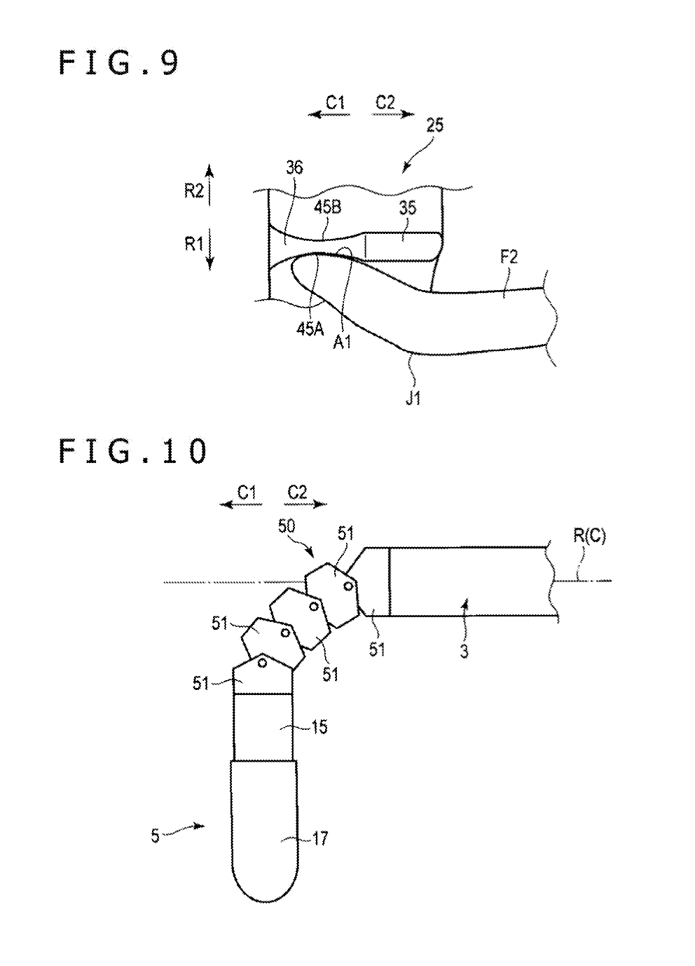

FIGS. 7 and 8 are views of the housing 2 of the treatment tool 1 that is held with one hand H0, i.e., the left hand in FIGS. 7 and 8. FIG. 7 is viewed from one side, i.e., the arrow W1 side, in the widthwise directions of the housing 7. FIG. 8 is viewed from a side of the axis of rotation R or the longitudinal axis C that is opposite the side where the grip 12 is positioned. FIGS. 7 and 8 illustrate the manner in which the index finger F2 is held against a second ridge 36 or one of the second ridges 36. FIG. 9 is a view illustrating the manner in which the index finger F2 abuts against a second ridge 36 or one of the second ridges 36, as viewed from the outer circumferential side of the rotary member 25.

As depicted in FIGS. 7 and 8, when the housing 2 is held by one hand H0, i.e., the left hand in FIGS. 7 and 8, the palm H1 and the thumb F1 are held against the grip 12 from the proximal-end side. The third finger F4 and the little finger F5 are placed on the force applying portion 22 of the handle 21, and the third finger f4 and/or the little finger F5 applies a manipulating force for opening or closing the handle 21 with respect to the grip 12 to the force applying portion 22. For bending the end effector 5 with respect to the shaft 3, the thumb F1 operates the bending dial or bending manipulation input portion 23. Each of the manipulating buttons 27A, 27B is pressed by the middle finger F3, which enters a manipulation input from each of the manipulating buttons 27A, 27B. If the manipulating button 27A or 27B is disposed on a portion of the outer surface of the housing 2 which faces one side in the widthwise directions, then the thumb F1 enters a manipulation input from the manipulating button 27A or 27B.

For adjusting the angular position of the end effector 5 about the axis of rotation R, the pad A1 of the index finger F2, i.e., a portion of the index finger F2 which extends from the distal interphalangeal (DIP) joint J1 toward the fingertip, is held against either one of the first ridges 35 of the rotary member 25. Then, the pad A1 of the index finger F2 applies a manipulating force for rotating the rotary member 25 about the axis of rotation R or the longitudinal axis C, to the rotary member 25. The shaft 3 and the end effector 5 are now rotated in unison with the rotary member 25 about the axis of rotation R with respect to the housing 2.

In a treatment using the treatment tool 1, a force may act on the end effector 5 while the end effector 5 is being bent with respect to the shaft 3 or the axis of rotation R. Since the force acts on the end effector 5 at a position spaced from the central axis or the axis of rotation R of the shaft 3, an angular moment may be produced around the axis of rotation R, i.e., the central axis of the shaft 3, by the force acting on the end effector 5. When an angular moment is produced around the axis of rotation R by the force acting on the end effector 5, the operator extends the index finger F2 from the state in which the pad A1 of the index finger F2 is in abutment against a first ridge 35 or one of the first ridges 35 to move the pad A1 of the index finger F2 toward the distal-end side, i.e., the arrow C1 side, of the treatment tool 1. Then, as illustrated in FIGS. 7 through 9, the operator brings the fingertip portion, i.e., the distal-end portion, of the pad A1 of the index finger F2 into abutment against one of the second ridges 36.

Each of the second ridges 36 is contiguous to the distal-end side of a corresponding first ridge 35 or a corresponding one of the first ridges 35. With the index finger F2 being bent to a certain extent, a manipulating force tending to rotate the rotary member 25 is applied to a first ridge 35 or one of the first ridges 35 of the rotary member 25. Therefore, when the index finger F2 is extended from the state in which the pad A1 of the index finger F2 is in abutment against a first ridge 35 or one of the first ridges 35, the pad A1 of the index finger F2 abuts against a second ridge 36 or one of the second ridges 36. Therefore, at least one of the second ridges 36 is positioned in a range in which the index finger F2 can abut against it while the housing 2 is being held by the hand H0.

With a pistol-shaped treatment tool such as the treatment tool 1, the rotary member 25 is coupled to the distal-end side of the housing body 11, and the grip 12 is disposed in a region spaced from the axis of rotation R or the longitudinal axis C. The force applying portion 22 is positioned on the side of the axis of rotation R or the longitudinal axis C where the grip 12 is positioned, and is positioned on the distal-end side with respect to the grip 12. Consequently, while the housing 2 is being held by one hand H0 and the pad A1 of the index finger F2 is abutting against a second ridge 36 or one of the second ridges 36, the index finger F2 extends obliquely to the axis of rotation R or the longitudinal axis C, as viewed from one side or the arrow W1 side or the arrow W2 side in a direction that is traverse to the axis of rotation R and also to the directions along which the grip 2 extends, i.e., the directions indicated by the arrows Y1, Y2.

While the housing 2 is being held by one hand H0 and the pad A1 of the index finger F2 is abutting against a second ridge 36 or one of the second ridges 36, the index finger F2 is held against the slanted surface 46A or 46B and extends along the slanted surface 46A or 46B. If the housing 2 is held by the left hand, then the index finger F2 extends along the slanted surface 46A that faces one side or the arrow W1 side in the widthwise directions. If the housing 2 is held by the right hand, then the index finger F2 extends along the slanted surface 46B that faces the other side or the arrow W2 side in the widthwise directions. Therefore, while the housing 2 is being held by one hand H0 and the pad A1 of the index finger F2 is abutting against a second ridge 36 or one of the second ridges 36, the index finger F2 extends obliquely to the axis of rotation R, as viewed from a side of the axis of rotation R which is opposite the side where the grip 12 is positioned. As the slanted surfaces 46A, 46B are formed as described hereinbefore, the index finger F2 is inclined to the axis of rotation R or the longitudinal axis C so as to be directed toward the inside in a widthwise direction of the treatment tool 1 gradually toward the fingertip portion thereof.

While the pad A1 of the index finger F2 is abutting against a second ridge 36 or one of the second ridges 36, the portion of the index finger F2 that is closer to its base than a distal interphalangeal joint or first joint J1 thereof extends obliquely to the axis of rotation R at a tilt angle α1 or first tilt angle α1, as viewed from the side of the axis of rotation R which is opposite the side where the grip 12 is positioned. At this time, the portion of the index finger F2 that is closer to its fingertip than the distal interphalangeal joint or DIP joint J1 thereof extends obliquely to the axis of rotation R at a tilt angle α2 or second tilt angle α2 that is larger than the tilt angle al or first tilt angle α1, as viewed from the side of the axis of rotation R which is opposite the side where the grip 12 is positioned. Consequently, while the pad A1 of the index finger F2 is abutting against a second ridge 36 or one of the second ridges 36, the portion of the index finger F2 that is closer to its fingertip than the distal interphalangeal joint J1 thereof is bent more to the inside in a widthwise direction of the treatment tool 1 than the portion of the index finger F2 that is closer to its base than the distal interphalangeal joint J1. Each of the tilt angles α1, α2 is an acute angle.

When an angular moment is produced toward one side, e.g., the arrow R1 side, around the axis of rotation R by a force acting on the end effector 5, as described hereinbefore, the pad A1 of the index finger F2 abutting against a second ridge 36 or one of the second ridges 36 applies a restraining force tending to restrain the rotation of the shaft 3 and the end effector 5 due to the angular moment. The second distance D2 from the axis of rotation R or the longitudinal axis C to the protrusive end T2 of each of the second ridges 36 is larger than the first distance D1 from the axis of rotation R to the protrusive end T1 of each of the first ridges 35. Accordingly, when the restraining force is applied to a second ridge 36 or one of the second ridges 36, a torque tending to cancel the rotation of the shaft 3 and the end effector 5 due to the angular moment, i.e., the product of the restraining force and the distance from the axis of rotation R to the position where the restraining force is applied, is large. Therefore, even when the angular moment is produced toward one side, e.g., the arrow R1 side, around the axis of rotation R by the force acting on the end effector 5, the restraining force applied to a second ridge 36 or one of the second ridges 36 effectively prevents the end effector 5 and the shaft 3 from being rotated about the axis of rotation R. The performance of a treatment that is performed while the treatment target is gripped between the gripping members 16, 17 is thus adequately maintained.

Each of the second ridges 36 has the recesses 45A, 45B defined therein that are recessed around the axis of rotation R, i.e., in the circumferential directions of the rotary member 25. Each of the recesses 45A, 45B is shaped to fit over the pad of an index finger. Therefore, on a second ridge 36 or one of the second ridges 36 against which the pad A1 of the index finger F2 is held, the fingertip portion of the pad A1 is fitted in the recess 45A or 45B. If the housing 2 is held by the left hand, then the pad A1 is fitted in the recess 45A. If the housing 2 is held by the right hand, then the pad A1 is fitted in the recess 45B. As the fingertip portion of the pad A1 is fitted in the recess 45A or 45B, it is easy to apply a restraining force to the second ridge 36 or one of the second ridges 36, and the restraining force can be increased. Since the second ridges 36 are disposed symmetrically around the axis of rotation R, regardless of which angular position the end effector 5 is in around the axis of rotation R, at least one of the second ridges 36 or any one of the second ridges 36 is in a position where it is easy for the pad A1 of the index finger F2 to apply a restraining force thereto.

Each of the second ridges 36 is contiguous to the distal-end side of a corresponding one of the first ridges 35, and is positioned on the distal-end side with respect to the first ridge 35. Therefore, when the pad A1 of the index finger F2 is held against a first ridge 35 or one of the first ridges 35 to rotate the rotary member 25 around the axis of rotation R, a manipulation for rotating the rotary member 25 is not obstructed by the second ridge 36.

While the housing 2 is being held by one hand H0 and the fingertip portion of the pad A1 of the index finger F2 is abutting against one of the second ridges 36, the pad A1 extends toward the proximal-end side in a second valley 43 or a corresponding one of valleys 43 adjacent on one side or the arrow R1 side around the axis of rotation R to the second ridge 36 or the one of the second ridges 36 against which the fingertip portion of the pad A1 is abutting. The distal interphalangeal joint J1 or a nearby portion thereof, e.g., the portion of the pad A1 on the base side, is held against one of the first ridges 35. A second ridge 36 or one of the second ridges 36 which is contiguous to the distal-end side of the first ridge 35 or the corresponding one of the first ridges 35 against which the distal interphalangeal joint J1 or the nearby portion thereof is held is disposed adjacent on one side around the axis of rotation R to the second ridge 36 or the one of the second ridges 36 against which the fingertip portion of the pad A1 is held. In other words, the second ridge 36 or the one of the second ridges 36 against which the fingertip portion of the pad A1 of the index finger F2 is held and the first ridge 35 or the corresponding one of the first ridges 35 against which the distal interphalangeal joint J1 or the nearby portion thereof, e.g., the proximal-end-side portion of the pad A1, is held are disposed adjacent to each other.

When an angular moment is produced toward the other side, e.g., the arrow R2 side, around the axis of rotation R by a force acting on the end effector 5, the distal interphalangeal joint or first joint J1 or a nearby portion thereof abutting against a first ridge 35 or one of first ridges 35 applies a restraining force tending to restrain the rotation of the shaft 3 and the end effector 5 due to the angular moment. With the distal interphalangeal joint J1 or the nearby portion thereof abutting against the first ridge 35 or the one of first ridges 35, it is easy to apply a restraining force to the first ridge 35 or the corresponding one of the first ridges 35, and the restraining force acting on the first ridge 35 or the corresponding one of the first ridges 35 can be increased. Accordingly, when the restraining force is applied to a first ridge 35 or a corresponding one of the first ridges 35, a torque tending to cancel the rotation of the shaft 3 and the end effector 5 due to the angular moment, i.e., the product of the restraining force and the distance from the axis of rotation R to the position where the restraining force is applied, is large. Therefore, even when the angular moment is produced toward the other side, e.g., the arrow R2 side, around the axis of rotation R by the force acting on the end effector 5, the restraining force applied to a first ridge 35 or one of the first ridges 35 effectively prevents the end effector 5 and the shaft 3 from being rotated about the axis of rotation R. The performance of a treatment that is performed while the treatment target is gripped between the gripping members 16, 17 is thus adequately maintained.

### Modifications

In the embodiment described hereinbefore, the rotary member 25 has the eight first ridges 35 and the eight second ridges 36. However, the number of first ridges 35 and the number of second ridges 36 are not limited to these numerical values. The rotary member may have nine or more first ridges 35 and nine or more second ridges 36, or seven or less first ridges 35 and seven or less second ridges 36. Alternatively, the rotary member may have only one first ridge 35 and only one second ridge 36. In either case, the number of first ridges 35 and the number of second ridges 36 are the same as each other, and each of the second ridges 36 is contiguous to the distal-end side of a corresponding one of the first ridges 35.

The treatment tool in which an angular moment is produced around the axis of rotation R by a force acting on the end effector 5 is not limited to the treatment tool 1 with the bendable joint 18 according to the first embodiment. According to a first modification illustrated in FIG. 10, a bendable portion 50 may be disposed in place of the bendable joint 18 on the distal-end side of the shaft 3. The bendable portion 50 includes a plurality of arrayed bend elements 51 each angularly movably coupled to an adjacent bend element or elements 51 or a corresponding one or two of the bend elements 51. A bending manipulation input portion is disposed on the housing 2 in place of the bending dial or bending manipulation input portion 23. The bendable portion 50 is actuated by operating the bending manipulation input portion. When the bendable portion 50 is actuated, the end effector 5 that includes the bendable portion 50 is bent with respect to the shaft 3 or the axis of rotation R. When a force acts on the end effector 5 that is being bent with respect to the shaft 3, since the force acts thereon at a position spaced from the axis of rotation R, as described hereinbefore, an angular moment may possibly be produced around the axis of rotation R or the central axis of the shaft 3. According to the present modification, since the treatment tool has the rotary member 25 that is similar to the rotary member of the first embodiment, even if an angular moment is produced around the axis of rotation R by the force acting on the end effector 5, a restraining force is applied to a second ridge 36 or one of the second ridges 36 or a first ridge 35 or one of the first ridges 35. Consequently, the end effector 5 and the shaft 3 are effectively prevented from being rotated around the axis of rotation R.

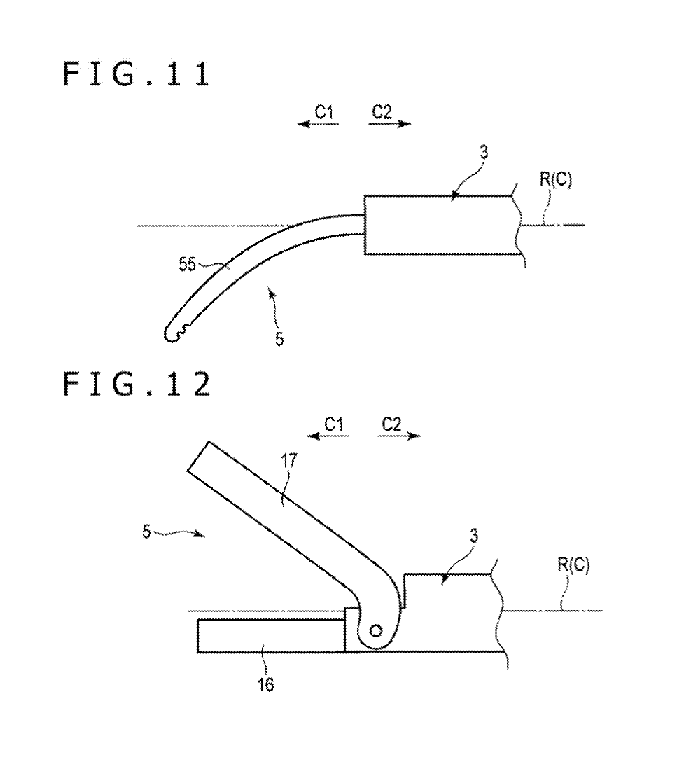

According to a second modification illustrated in FIG. 11, an end effector 5 projects toward the distal-end side from the distal end of the shaft 3. The end effector 5 includes a curved extension 55 extending so as to be bendable with respect to the shaft 3 or the axis of rotation R. When a force acts on the end effector 5 at a region on the distal-end side from the bendable portion 55, since the force acts thereon at a position spaced from the axis of rotation R, as described hereinbefore, an angular moment may possibly be produced around the axis of rotation R or the central axis of the shaft 3. According to the present modification, since the treatment tool has the rotary member 25 that is similar to the rotary member of the first embodiment, even if an angular moment is produced around the axis of rotation R by the force acting on the end effector 5, a restraining force is applied to a second ridge 36 or one of the second ridges 36 or a first ridge 35 or one of the first ridges 35. Consequently, the end effector 5 and the shaft 3 are effectively prevented from being rotated around the axis of rotation R.

According to a third modification illustrated in FIG. 12, as with the first embodiment, a pair of gripping members 16, 17 are mounted on an end effector 5. According to the present modification, the treatment tool 1 is devoid of either of the bendable joint 18, the bendable portion 50, and the curved extension 55. The treatment tool 1 with the gripping members 16, 17 may perform a treatment while the space between the gripping members 16, 17 is being open. While the space between the gripping members 16, 17 is being open, if a force acts on a gripping member, e.g., the second gripping member 17, angularly movably mounted on the shaft 3, since the force acts thereon at a position spaced from the axis of rotation R, as described hereinbefore, an angular moment may possibly be produced around the axis of rotation R or the central axis of the shaft 3. According to the present modification, since the treatment tool has the rotary member 25 that is similar to the rotary member of the first embodiment, even if an angular moment is produced around the axis of rotation R by the force acting on the end effector 5, a restraining force is applied to a second ridge 36 or one of the second ridges 36 or a first ridge 35 or one of the first ridges 35. Consequently, the end effector 5 and the shaft 3 are effectively prevented from being rotated around the axis of rotation R.

According to the embodiment, etc. described hereinbefore, the treatment tool 1 includes the housing 2 that can be held, the shaft 3 that is rotatable around the axis of rotation R with respect to the housing 2, the end effector 5 disposed on the distal-end portion of the shaft 3, and the rotary member 25 that is rotatable in unison with the shaft 3 and the end effector 5 around the axis of rotation R. The rotary member 25 has the first ridge 35 projecting toward an outer circumferential side and having the first distance D1 from the axis of rotation R to the protrusive end T1 thereof, and the second ridge 36 contiguous to a distal-end side of the first ridge 35, projecting toward the outer circumferential side, and having the second distance D2 from the axis of rotation R to the protrusive end T2 thereof, the second distance D2 being larger than the first distance D1.

### REFERENCE EXAMPLES

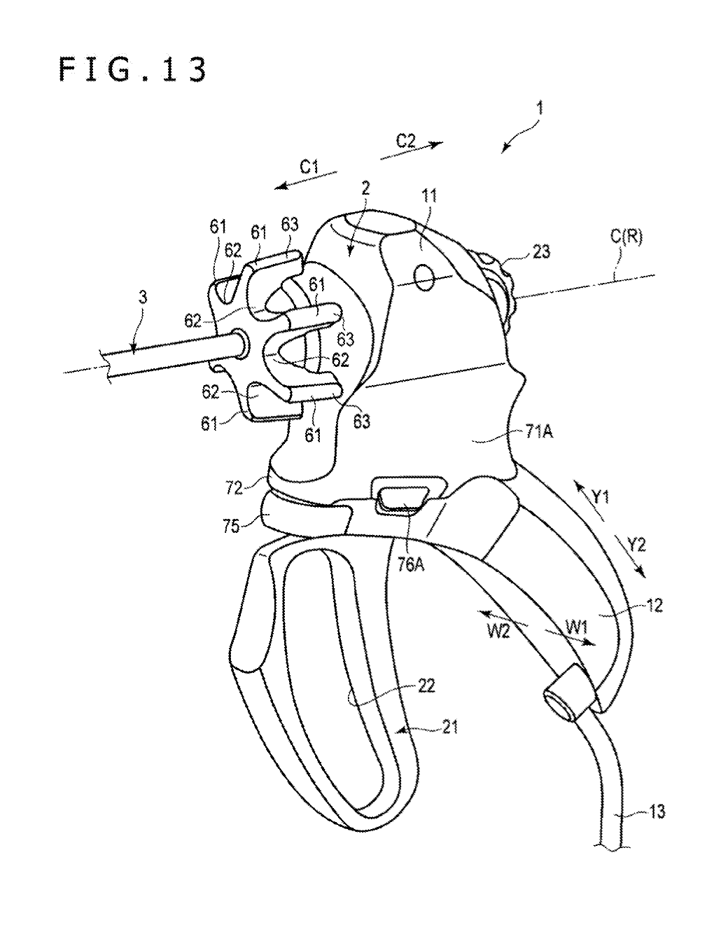

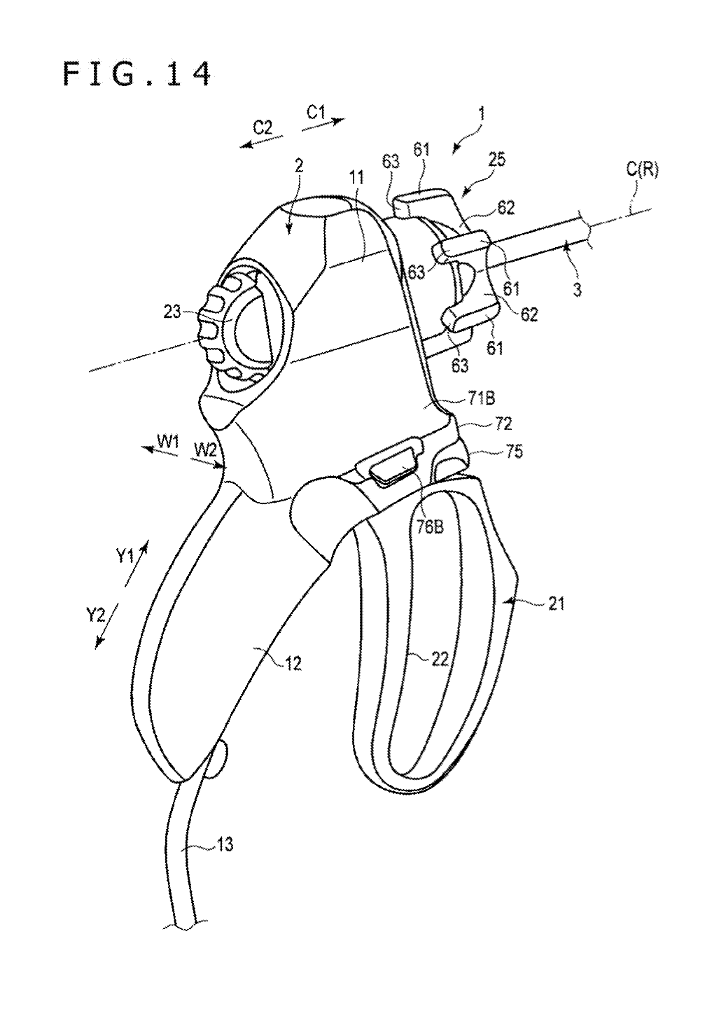

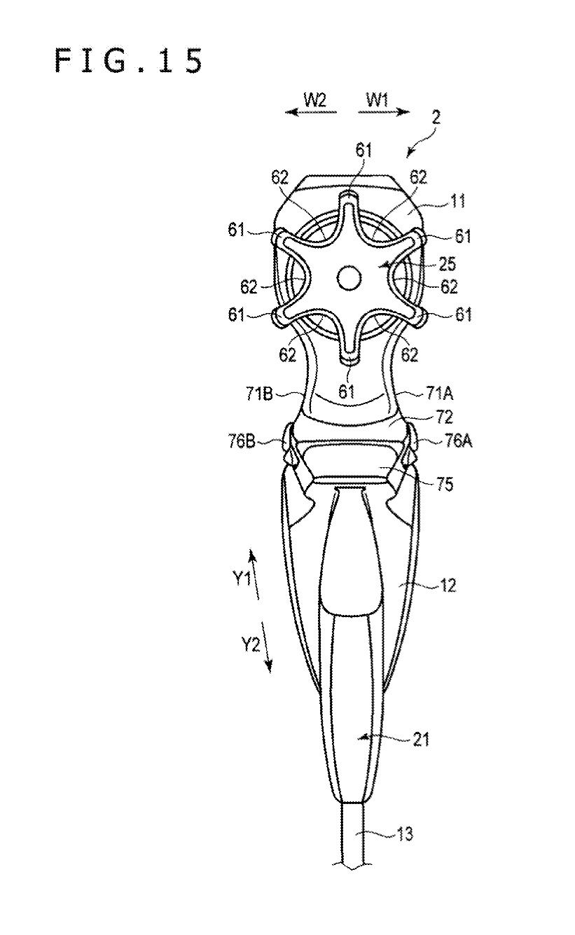

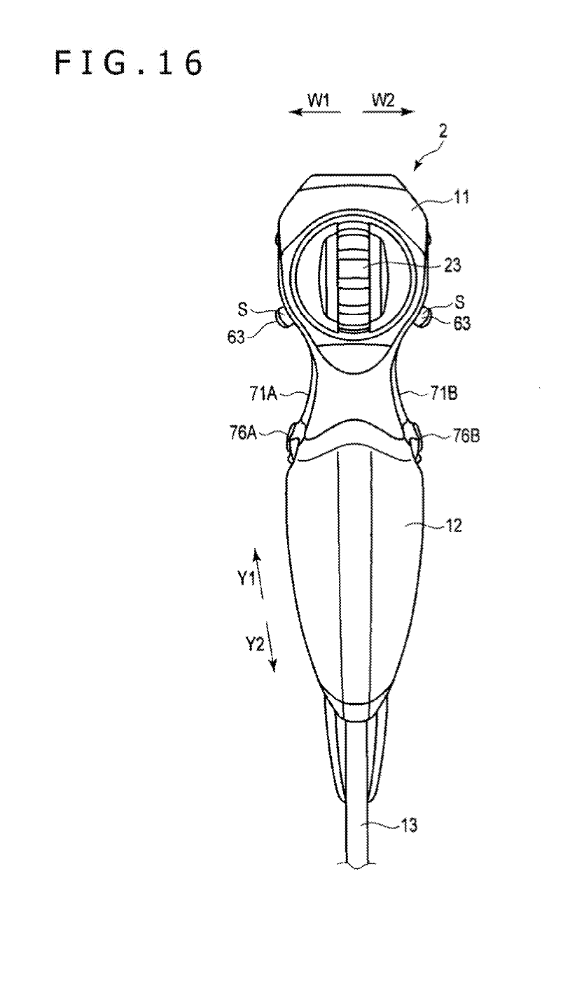

FIGS. 13 through 16 are views of a housing 2 and nearby structures of a treatment tool 1 according to a reference example. FIGS. 13 and 14 are perspective views as viewed from directions that are different from each other. FIG. 15 illustrates the treatment tool 1 as viewed from the distal-end side, i.e., the arrow C1 side. FIG. 16 illustrates the treatment tool 1 as viewed from the proximal-end side, i.e., the arrow C2 side. According to the present reference example, as with the first embodiment, the treatment tool 1 includes a housing 2, a shaft 3, and an end effector 5. The end effector 5 is bendable with respect to the shaft 3 or an axis of rotation Rat a bendable joint 18. The space between a pair of gripping members 16, 17 of the end effector 5 can be opened and closed. The shaft 3 and the end effector 5 are rotatable in unison with each other around the axis of rotation R, i.e., the central axis of the shaft 3, with respect to the housing 2.

According to the present reference example, as with the first embodiment, the treatment tool 1 is pistol-shaped, and the housing 2 includes a housing body 11 and a grip 12. According to the present reference example, too, a rotary member 25 is coupled to the distal-end side of the housing body 11, and the grip 12 is disposed in a region spaced from the axis of rotation R or a longitudinal axis C. According to the present reference example, the grip 12 also extends along directions transverse to the longitudinal axis C, i.e., the directions indicated by the arrows Y1, Y2. According to the present reference example, a handle 21 is also angularly movably mounted on the housing 2. A force applying portion 22 is positioned on a side of the axis of rotation R, i.e., the longitudinal axis C, where the grip 12 is positioned, and on the distal-end side with respect to the grip 12. According to the present reference example, a bending dial 23 is also mounted as a bending manipulation input portion on the housing 2.

According to the present reference example, a plurality of (six in the present reference example) ridges 61 are mounted on an outer circumferential surface of a rotary member 25 and project toward an outer circumferential side. The ridges 61 are exposed outwardly and spaced apart from each other around the axis of rotation R, i.e., the longitudinal axis C. Each of the ridges 61 and another ridge 61 or a corresponding one of the ridges 61 that is disposed adjacent to the ridge 61 on one side around the axis of rotation R define therebetween a valley 62 or a corresponding one of valleys 62 that is recessed toward the inner circumferential side. In other words, on the outer circumferential surface of the rotary member 25, the ridges 62 and the valleys 62 are alternately disposed around the axis of rotation R, i.e., the longitudinal axis C.

Each of the ridges 61 includes a proximal-end-side portion 63 or a corresponding one of proximal-end-side portions 63 extending farther toward the proximal-end side from the distal end of the housing body 11, i.e., the proximal end of each of the valleys 62. Therefore, the proximal end of each of the ridges 61 is positioned on the proximal-end side with respect to the distal end of the housing body 11, i.e., the proximal end of each of the valleys 62. Each of the proximal-end-side portions or projecting ridge portions 63 is disposed on an outer circumferential side of a distal-end portion of the housing body 11, with a clearance S left between itself and the outer surface of the housing body 11. Therefore, the proximal-end-side portion 63 or the corresponding one of the proximal-end-side portions 63 of each of the ridges 61 is kept out of contact with the outer surface of the housing body 11. The distance from the axis of rotation R or the longitudinal axis C to a protrusive end of each of the ridges 61 is substantially uniform from the proximal end or the proximal-end-side portion 63 or the corresponding one of the proximal-end-side portions 63 of each of the ridges 61 to the distal end of the ridge 61.

In the case where each of the ridges 61 includes the proximal-end-side portion 63 or the corresponding one of the proximal-end-side portions 63, as described hereinbefore, the dimension of each of the ridges 61 in the directions along the axis of rotation R or the longitudinal axis C is large. Consequently, the operator finds it easy to apply a manipulating force tending to rotate the shaft 3 and the end effector 5 around the axis of rotation R to a ridge 61 or one of the ridges 61 of the rotary member 25.

The outer surface of the housing 2 has a first side face 71A facing one side, i.e., the arrow W1 side, in the widthwise directions and a second side face 71B facing the other side, i.e., the arrow W2 side, in the widthwise directions. The outer surface of the housing 2 also has a mount surface 72 or a distal-end surface that faces the distal-end side at a position between the force applying portion 22 of the handle 21 and the rotary member 25. According to the present reference example, a first manipulating button 75 is disposed on the mount surface 72. According to the present reference example, since each of the ridges 61 includes the proximal-end-side portion 63 or the corresponding one of the proximal-end-side portions 63, as described hereinbefore, the mount surface 72 may possibly fail to provide an enough space to have a plurality of manipulating buttons, e.g., the first manipulating button 75, etc. thereon.

According to the reference example, therefore, only the single first manipulating button 75 is mounted on the mount surface 72. A second manipulating button 76A is mounted on the first side face 71A. A third manipulating button 76B is mounted on the second side face 71B. The second manipulating button 76A is disposed on the first side face 71A at a position between the grip 12 and the rotary member 25. The third manipulating button 76B is disposed on the second side face 71B at a position between the grip 12 and the rotary member 25.

According to the present reference example, when a manipulation input is entered from the first manipulating button 75, the treatment tool 1 operates in a first mode of operation, causing a high-frequency current to flow through the treatment target gripped between the gripping members 16, 17. When a manipulation input is entered from the second manipulating button 76A or the third manipulating button 76B, the treatment tool 1 operates in a second mode of operation that is different from the first mode of operation, applying treatment energy different from a high-frequency current, i.e., ultraviolet vibrations, heat generated by a heat generator, or the like, to the gripped treatment target, or sticking a staple to the treatment target. According to the present reference example, in other words, both when a manipulation input is entered from the second manipulating button 76A and when a manipulation input is entered from the third manipulating button 76B, the treatment tool 1 operates in the second mode of operation.

According to the present reference example, while the housing 2 is being held by one hand H0, the middle finger F3 enters a manipulation input from the first manipulating button 75. While the housing 2 is being held by the right hand, the thumb F2 of the right hand enters a manipulation input from the second manipulating button 76A. While the housing 2 is being held by the right hand, no manipulation is made on the third manipulating button 76B. On the other hand, while the housing 2 is being held by the left hand, the thumb F2 of the left hand enters a manipulation input from the third manipulating button 76B. While the housing 2 is being held by the left hand, no manipulation is made on the second manipulating button 76A.

According to the present reference example, as described hereinbefore, when a manipulation input is entered from the second manipulating button 76A and a manipulation input is entered from the third manipulating button 76B, the treatment tool 1 operates in the second mode of operation. Therefore, both while the housing 2 is being held by the right hand and while the housing 2 is being held by the left hand, it is possible to operate the treatment tool 1 appropriately in the second mode of operation.

The disclosed technology is directed to a treatment tool comprises a housing and a sheath having respective proximal and distal ends. The sheath is configured to be attached to the housing via the proximal end. The sheath rotates around an axis of rotation with respect to the housing. An end effector is configured to be attached to the distal-end of the sheath. A rotary member is configured to be attached to the sheath. The rotary member rotates in unison with the sheath and the end effector around the axis of rotation. The rotary member having at least respective first and second ridges. The at least first ridge projects to an outer circumferential side and having a first distance from the axis of rotation to a protrusive end thereof. The at least second ridge is contiguous to a distal-end side of the at least first ridge projecting to the outer circumferential side and having a second distance from the axis of rotation to a protrusive end thereof. The second distance is longer than the first distance.

The at least second ridge includes a recess formed in the circumferential directions of the rotary member. The recess that defined in the at least second ridge is shaped so as to fit over the pad of an index finger. The at least first ridge is defined by a plurality of first ridges spaced apart from one another around the axis of rotation. The at least second ridge is defined by a plurality of second ridges spaced apart from one another around the axis of rotation. Each of the plurality of the second ridges is contiguous to a distal-end side of a corresponding one of the plurality of the first ridges. The plurality of the first ridges are disposed symmetrically around the axis of rotation and the plurality of the second ridges are disposed symmetrically around the axis of rotation. The treatment tool further comprises a grip configured to be attached to the housing and extends downwardly from the housing along a direction transverse to the axis of rotation. A pivotable handle is mounted on the housing. The pivotable handle having a force applying portion to which a manipulating force for pivoting the handle with respect to the grip is applicable. The force applying portion is positioned on a side of the axis of rotation where the grip is positioned and on the distal-end side with respect to the grip.

The housing is held by a hand, the palm and the thumb are held against the grip from the proximal-end side, the third finger and/or the little finger applies the manipulating force to the force applying portion of the handle, and the index finger applies a manipulating force for rotating the rotary member to the at least first ridge. The at least second ridge is positioned in a range in which the index finger is held there against while the housing is being held by the hand. The at least first ridge is defined by a plurality of first ridges spaced apart from one another around the axis of rotation. The at least second ridge is defined by a plurality of second ridges spaced apart from one another around the axis of rotation. Each of the plurality of the second ridges is contiguous to the distal-end side of a corresponding one of the plurality of the first ridges. The plurality of first ridges includes a first portion of the first ridges. The plurality of second ridges includes a first portion of the second ridges and a second portion of the second ridges. While the housing is being held by the hand and a fingertip portion of the pad of the index finger is held against the first portion of the second ridges, the distal interphalangeal joint of the index finger or a nearby portion thereof is held against the first portion of the first ridges, and the second portion of the second ridges that is contiguous to the distal-end side of the first portion of the first ridges against which the distal interphalangeal joint or the nearby portion thereof is held is disposed adjacent to the first portion of the second ridges against which the fingertip portion of the pad is held around the axis of rotation.

While the housing is being held by the hand and the index finger is being held against the at least second ridge, the index finger extends obliquely to the axis of rotation, as viewed from a side of the axis of rotation opposite to the side where the grip is positioned, and the index finger extends obliquely to the axis of rotation, as viewed from a side in a direction transverse to the axis of rotation and transverse to the direction in which the grip extends. The end effector is capable of being bended and/or curved with respect to the sheath. The rotary member has a face that defines a base of the at least first ridge and a base of the at least second ridge. The face includes an arcuate shape about the axis of rotation at its center as viewed in directions along the axis of rotation. A protruded distance by which the second ridge projects from a base thereof to the protrusive end thereof is longer than a protruded distance by which the first ridge projects from a base thereof to the protrusive end thereof.

While the embodiments and the modifications have been described hereinbefore, the present disclosure is not limited to the embodiments and the modifications. Various changes and modifications may be made therein without departing from the scope of the disclosure.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A treatment tool comprising:
a housing;
an elongated member having respective proximal and distal ends wherein the elongated member configured to be attached to the housing via the proximal end, the elongated member being rotatable around an axis of rotation with respect to the housing;
an end effector configured to be attached to the distal-end of the elongated member; and
a rotary member configured to be attached to the elongated member, the rotary member being rotatable in unison with the elongated member and the end effector around the axis of rotation, the rotary member having at least respective first and second ridges wherein the at least first ridge being projected to an outer circumferential side and having a first distance from the axis of rotation to a protrusive end thereof and the at least second ridge being contiguous to a distal-end side of the at least first ridge, projecting to the outer circumferential side and having a second distance from the axis of rotation to a protrusive end thereof, the second distance being longer than the first distance.

2. The treatment tool of claim 1, wherein the at least second ridge includes a recess formed in the circumferential directions of the rotary member.

3. The treatment tool of claim 2, wherein the recess that defined in the at least second ridge is shaped so as to fit over the pad of an index finger.

4. The treatment tool of claim 1, wherein the at least first ridge is defined by a plurality of first ridges spaced apart from one another around the axis of rotation, and the at least second ridge is defined by a plurality of second ridges spaced apart from one another around the axis of rotation; and
each of the plurality of the second ridges is contiguous to a distal-end side of a corresponding one of the plurality of the first ridges.

5. The treatment tool of claim 4, wherein the plurality of the first ridges are disposed symmetrically around the axis of rotation and the plurality of the second ridges are disposed symmetrically around the axis of rotation.

6. The treatment tool of claim 1, further comprising:
a grip configured to be attached to the housing and extended downwardly from the housing along a direction transverse to the axis of rotation; and
a pivotable handle is mounted on the housing, the pivotable handle having a force applying portion to which a manipulating force for pivoting the handle with respect to the grip is applicable, the force applying portion being positioned on a side of the axis of rotation where the grip is positioned, and on the distal-end side with respect to the grip.

7. The treatment tool of claim 6, wherein the housing is being held by a hand, the palm and the thumb are held against the grip from the proximal-end side, the third finger and/or the little finger applies the manipulating force to the force applying portion of the handle, and the index finger applies a manipulating force for rotating the rotary member to the at least first ridge.

8. The treatment tool of claim 7, wherein the at least second ridge is positioned in a range in which the index finger is held there against while the housing is being held by the hand.

9. The treatment tool of claim 8, wherein the at least first ridge is defined by a plurality of first ridges spaced apart from one another around the axis of rotation, and the at least second ridge is defined by a plurality of second ridges spaced apart from one another around the axis of rotation;
each of the plurality of the second ridges is contiguous to the distal-end side of a corresponding one of the plurality of the first ridges; and
the plurality of first ridges includes a first portion of the first ridges,
the plurality of second ridges includes a first portion of the second ridges and a second portion of the second ridges,
while the housing is being held by the hand and a fingertip portion of the pad of the index finger is held against the first portion of the second ridges, the distal interphalangeal joint of the index finger or a nearby portion thereof is held against the first portion of the first ridges, and the second portion of the second ridges that is contiguous to the distal-end side of the first portion of the first ridges against which the distal interphalangeal joint or the nearby portion thereof is held is disposed adjacent to the first portion of the second ridges against which the fingertip portion of the pad is held around the axis of rotation.

10. The treatment tool of claim 8, wherein while the housing is being held by the hand and the index finger is being held against the at least second ridge, the index finger extends obliquely to the axis of rotation, as viewed from a side of the axis of rotation opposite to the side where the grip is positioned, and the index finger extends obliquely to the axis of rotation, as viewed from a side in a direction transverse to the axis of rotation and transverse to the direction in which the grip extends.

11. The treatment tool of claim 1, wherein the end effector is capable of being bended and/or curved with respect to the elongated member.

12. The treatment tool of claim 1, wherein the rotary member has a face that defines a base of the at least first ridge and a base of the at least second ridge.

13. The treatment tool of claim 12, wherein the face includes an arcuate shape about the axis of rotation at its center as viewed in directions along the axis of rotation.

14. The treatment tool of claim 1, wherein a protruded distance by which the second ridge projects from a base thereof to the protrusive end thereof is longer than a protruded distance by which the first ridge projects from a base thereof to the protrusive end thereof.

15. The treatment tool of claim 1, wherein the elongated member is defined by a sheath.

16. The treatment tool of claim 1, wherein the elongated member is defined by a shaft.
